(19) United States
(12) Patent Application Publication
(10) Pub. No.: US 2010/0268065 A1
(43) Pub. Date: Oct. 21, 2010

(54) SYSTEMS AND METHODS FOR IMAGING A BLOOD VESSEL USING TEMPERATURE SENSITIVE MAGNETIC RESONANCE IMAGING

(76) Inventors: John Pile-Spellman, Pelham, NY (US); Erwin Lin, Whitestone, NY (US)

Correspondence Address: KENYON & KENYON LLP, ONE BROADWAY, NEW YORK, NY, 10004, US

(21) Appl. No.: 12/161,515

(22) PCT Filed: Jan. 22, 2007
(86) PCT No.: PCT/US07/02032

§ 371 Date: Jun. 9, 2010

Related U.S. Application Data
(60) Provisional application No. 60/761,773, filed on Jan. 25, 2006.

(51) Int. Cl. A61B 5/055

(52) U.S. Cl. 600/419

(57) ABSTRACT

A method for producing an image of a blood vessel in a patient utilizing temperature sensitive MRI measurement. The method includes introducing a fluid in a blood vessel, obtaining magnetic resonance information from the blood vessel, and determining a magnetic resonance parameter using the magnetic resonance information. The method further includes using the magnetic resonance parameter to determine a temperature differential in the blood vessel and producing an image of the blood vessel based on the temperature differential. Systems for producing an image of a blood vessel in a patient using temperature sensitive MRI measurements are also provided.

### CROSS-REFERENCE TO RELATED APPLICATIONS

The present application claims the benefit of and priority to International Patent Application No. PCT/US07/02032, filed 22 Jan. 2007, which claims the benefit of and priority to U.S. Provisional Patent Application No. 60/761,773, filed 25 Jan. 2006, both of which are expressly incorporated herein in their entireties by reference thereto.

The present application is related to co-pending applications “Systems and Methods for Determining a Cardiovascular Parameter Using Temperature Sensitive Magnetic Resonance Imaging,” filed herewith and “Systems and Methods for Determining Metabolic Rate Using Temperature Sensitive Magnetic Resonance. Imaging,” filed herewith. Both applications are incorporated by reference herein.

### FIELD OF THE INVENTION

The present invention relates to methods and systems for producing an image of a blood vessel based on a temperature differential determined from information obtained by magnetic resonance imaging.

### BACKGROUND

Angiography is the visualization of blood vessels and can be accomplished with various diagnostic imaging modalities. Conventional x-ray angiography requires injection of iodinated contrast material into a blood vessel through an intra-arterial or intravenous catheter followed by sequential x-ray exposures using conventional film cassettes or digital technology. Conventional x-ray angiography is an invasive procedure and the injection of iodinated contrast material can be associated with adverse reactions including severe allergic reactions and anaphylaxis. Recently, computerized tomography (CT) angiography has begun to replace conventional x-ray angiography. CT angiography has spatial and contrast resolution that is near that of conventional angiography, it is less invasive (only requires an intravenous injection of contrast material) and it allows for multiplanar reconstruction. However, CT angiography still requires the use of x-rays. In addition, because they require iodinated contrast agents, both conventional x-ray angiography and CT angiography cannot be readily repeated. Diagnostic ultrasound with Doppler or color flow imaging can be used to obtain angiographic images of major blood vessels. However, ultrasound angiography has limited spatial resolution, limited depth of penetration into the body and does not readily allow multiplanar reconstruction. In addition, ultrasound angiography cannot visualize the cerebral vasculature,

Magnetic Resonance Angiography (MRA) is a non-invasive technique that does not use ionizing radiation, does not use iodinated contrast material and allows for multiplanar reconstruction. There are two general categories of MRA: contrast enhanced and non-contrast enhanced. Contrast enhanced MRA is performed by imaging after intravenous administration of gadolinium-containing contrast agents. Although these contrast agents are safer than iodinated agents, they still carry the risk of adverse reactions. Contrast enhanced MRA images are obtained during a narrow window of time when the concentration of contrast agent in the vascular space is near its peak and the concentration of contrast agent in the extravascular space is minimal. Advantages of contrast enhanced MRA (compared with non-contrast enhanced MRA) include image signal based on the concentration of contrast agent in the vessel lumen similar to conventional x-ray angiography and CT angiography, higher signal-to-noise ratio and better spatial and contrast resolution. When using gadolinium-based techniques, only a single dose of gadolinium contrast agent can typically be administered at any one time due to safety concerns. In addition, gadolinium contrast agents are expensive. Since the MR signal of contrast enhanced MRA is derived only from the vessel lumen, the vessel wall (or edge) may not be visualized or may be ill-defined. Visualization of the vessel wall may be important for diagnosis of vascular disease, especially small vessel disease, and tracking of vessel wall motion can be used for image gating.

Non-contrast enhanced MRA can be performed using time-of-flight techniques or phase contrast techniques. Time-of-flight techniques rely on the motion of flowing blood to provide signal differences between blood vessels and surrounding soft tissues. Phase contrast techniques rely on motion-induced phase changes in the presence of magnetic field gradients to provide signal differences between blood vessels and surrounding soft tissues. An advantage of the time-of-flight and phase contrast techniques (compared with contrast enhanced techniques) is that they can be performed repeatedly in seconds to minutes without any additional risk. In general however, time-of-flight and phase contrast techniques have lower signal-to-noise and lower spatial resolution than contrast enhanced techniques and, like contrast-enhanced MRA, the edge of blood vessels may not be well-defined. Furthermore, time-of-flight and phase contrast techniques suffer from artifacts related to differences in flow velocity across the lumen of a blood vessel and they do not image blood vessels based on the presence of an intravascular agent.

A need, therefore, exists for an improved MRA technique that provides higher resolution than prior methods, is repeatable, and does not carry the risk of adverse reactions.

### SUMMARY OF THE INVENTION

Systems and methods of imaging a blood vessel using temperature sensitive MRI are provided. In an embodiment, the present invention provides a method for producing an image of a blood vessel of a patient based on a temperature differential of flowing blood within the vessel determined from information obtained by MRI. The method includes introducing a fluid into a cardiovascular system of the patient and obtaining magnetic resonance information from the blood vessel. The method further includes determining a magnetic resonance parameter in the blood vessel using the magnetic resonance information and determining a temperature differential in the blood vessel using the magnetic resonance parameter. The method further includes producing an image of the blood vessel in which a brightness or a color of pixels therein is based on the temperature differential determined using the magnetic resonance parameter. For example, a threshold temperature differential can be used to display flow in a vessel lumen compared with absence of flow in surrounding tissues using a fixed brightness or fixed color. Alternatively, a temperature differential determined over time can be used to display flow in a vessel lumen such that a brightness or color may reflect both temperature differentials and local flow characteristics.

In an embodiment, the present invention provides a machine-readable medium having stored thereon a plurality of executable instructions, when executed by a processor performs obtaining magnetic resonance information from a blood vessel of a patient after introduction of fluid into a cardiovascular system of the patient and determining a magnetic resonance parameter in the blood vessel using the magnetic resonance information. The plurality of executable instructions further performs determining a temperature differential in the blood vessel using the magnetic resonance parameter and producing an image of the blood vessel in which a brightness or a color of pixels therein is determined by the temperature differential.

### BRIEF DESCRIPTION OF THE DRAWINGS

FIG. 1 is a flow diagram that illustrates an embodiment of a method of producing an image of a blood vessel using temperature sensitive MRI.

FIG. 2 illustrates an embodiment of a system to control the temperature and flow of fluid introduced into a patient.

FIG. 3 is a block diagram that depicts an embodiment of a user computing device.

FIG. 4 is a block diagram that depicts an embodiment of a network architecture.

### DETAILED DESCRIPTION OF THE INVENTION

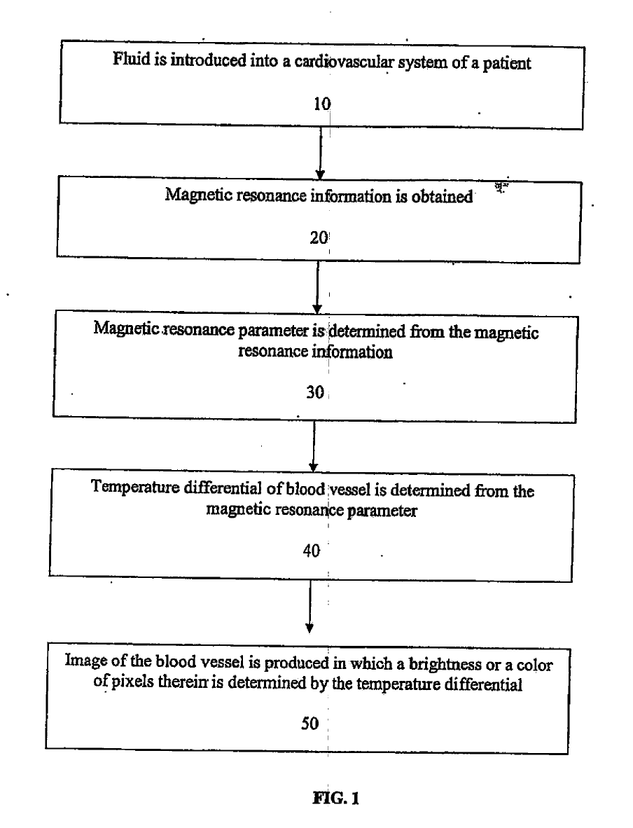

In an embodiment, the present invention provides a method for producing an image of a blood vessel of a patient based on a temperature differential of flowing blood within the vessel determined from information obtained by MRI. Specifically, referring to FIG. 1, a method for producing an image of a blood vessel comprises introducing a fluid into a cardiovascular system of a patient (10) and then obtaining magnetic resonance information from the blood vessel of the patient (20). A magnetic resonance parameter is determined using the magnetic resonance information (30) and a temperature differential in the blood vessel is determined using the magnetic resonance parameter (40). Based on the temperature differential, an image of the blood vessel is produced in which a brightness or a color of pixels therein is determined by the temperature differential (50).

The blood vessel can be a part of the vasculature of a patient including an artery, vein, capillary or combination thereof. The artery or vein can be a central or peripheral artery or vein. Non-limiting examples of blood vessels include the carotid artery, internal jugular vein, inferior or superior vena cava, aorta, pulmonary artery and vein, illiac artery and vein, femoral artery and vein, popliteal artery and vein, anterior tibial artery and vein, posterior tibial artery and vein, and peroneal artery and vein. Images of a single blood vessel or multiple blood vessels can be obtained according to methods of the present invention.

Referring again to FIG. 1, with respect to introducing a fluid into a blood vessel of a patient (10), the fluid is any biologically compatible fluid that can perfuse the portion of the body. For example, the fluid may be water, blood or a saline solution. The fluid can be introduced over any time frame at any rate sufficient to induce temperature changes that can be effectively imaged. For example, the fluid may be introduced at a constant rate over a period of seconds, such as, for example, a bolus injection where the shape of the input is a square wave. Alternatively, the fluid may be introduced over a period of minutes, where the shape of the input is a desired function of time including a sinusoidal function. Furthermore, the shape of the input may be designed to optimize the arterial input function of the blood vessel being imaged and thereby simplify calculations.

The fluid can be introduced in any manner such that the fluid can perfuse the blood vessel being imaged and induce temperature changes that can be effectively imaged. For example, the fluid can be injected intravenously or intra-arterially or introduced as a gas in the lungs via inhalation. Further, the fluid can be introduced at a site local or distant to the blood vessel being imaged. For example, the fluid may be injected into a peripheral vein using a conventional intravenous line, into a central vein using a central venous line or through a catheter or needle in a central or peripheral artery that supplies the blood vessel being imaged. The temperature of the introduced fluid can be above or below body temperature. Further, the temperature of the introduced fluid may have a uniform constant temperature below or above body temperature or can vary over time and include temperatures above and below body temperature. For example, the introduced fluid may vary over time when the injection site is remote from the tissue of interest, such as a peripheral vein, and the profile of the injected fluid changes after passing through the heart and pulmonary circulation. Using an injection with a time-varying temperature may reduce such changes. A constant temperature injection may be used, for example, when the injection site is closer to the tissue of interest, such as a central artery, and the profile of the injected fluid does not change as readily.

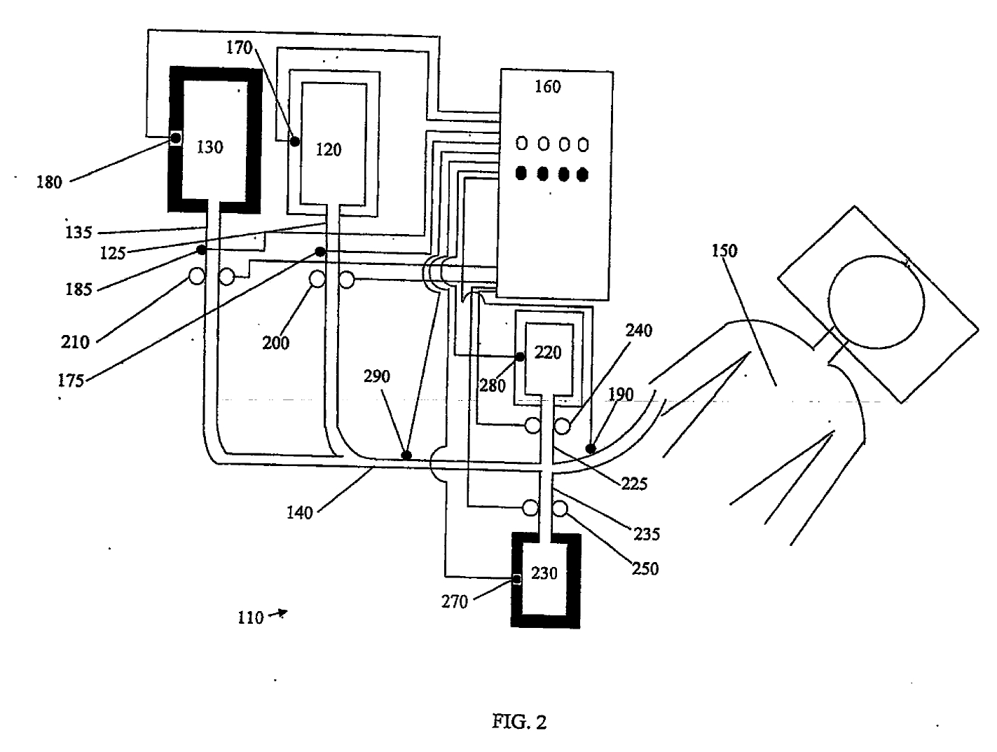

A system can be used for controlling the temperature of the fluid that is introduced into the patient by combining fluids having two different temperatures and introducing the combined fluid into the patient. Referring to FIG. 2, in an embodiment, such a system 110 includes first reservoir 120 containing a first fluid at a temperature below body temperature and second reservoir 130 containing a second fluid at a temperature above body temperature. First and second reservoirs 120 and 130 are in fluid communication with respective first and second fluid lines 125 and 135, which, in turn, are in fluid communication with a convergent line 140. First and second lines 125 and 135 can converge with convergent line 140 via a Y-connector, for example, such that the fluid outflow of reservoirs 120 and 130 is combined into a single fluid line. In this embodiment, system 110 further comprises third reservoir 220 containing a third fluid at a temperature below body temperature and fourth reservoir 230 containing a fourth fluid at a temperature above body temperature. Third and fourth reservoirs 220 and 230 are in fluid communication with respective third and fourth fluid lines 225 and 235, which, in turn, are in fluid communication with convergent line 140. Convergent line 140 is insertable into a blood vessel of a patient 150 either directly or indirectly, via a catheter attached to the distal end of convergent line 140.

In this embodiment, system 110 further comprises first reservoir temperature sensor 170 in communication with first reservoir 120 and first line temperature sensor 175 in communication with first fluid line 125. System 110 further comprises second reservoir temperature sensor 180 in communication with second reservoir 130 and second line temperature sensor 185 in communication with second fluid line 135. System 110 further comprises third reservoir temperature sensor 280 in communication with third reservoir 220 and fourth reservoir temperature sensor 270 in communication with fourth reservoir 230. In addition, system 110 comprises convergent line temperature sensors 190 and 290. System 110 further comprises controller 160 for controlling the flow of first, second, third and fourth fluids from respective first, second, third and fourth reservoirs 120, 130, 220, and 230 . Specifically, in an embodiment, controller 160 is in communication with sensors 170, 180, 175, 185, 190, 270, 280 and 290. Controller 160 is also in communication with first pump 200, second pump 210, third pump 240 and fourth pump 250 which, in turn, are in communication with first fluid line 125, second fluid line 135, third fluid line 225 and fourth fluid line 235, respectively. A non-limiting example of first, second, third and fourth pumps 200, 210, 240 and 250 are power injectors. In certain embodiments, a system does not include third and fourth pumps. In certain embodiments, a system does not include a third and fourth pump. In order to control the flow of first and second fluids, controller 160 receives temperature input signals from sensors 170, 180, 175, and 185 regarding the temperature of the first and second fluids and accordingly sends out a control signal to pumps 200 and 210 to adjust the flow rate of the fluids. Likewise, in order to control the flow of third and fourth fluids, controller 160 receives temperature input signals from sensors 280 and 270 regarding the temperature of the third and fourth fluids and accordingly sends out a control signal to pumps 240 and 250 to adjust the flow rate of the fluids. Controller 160 may be computerized and the flow rate of first and second fluids exiting respective first and second reservoirs 120 and 130 can be varied in accordance, for example, with a look-up table or an algorithm to achieve a desired temperature variation of the introduced combined fluid. Temperature readings from the convergent line temperature sensors 190 and 290 can be used to confirm the expected temperature in convergent line 140 as determined, for example, from the look-up table or the algorithm. Controller 160 may be computerized and may introduce additional fluid from third and fourth reservoirs 220 and 230 in accordance, for example, with a look-up table or an algorithm to make adjustments to achieve the desired temperature variation of the introduced fluid or to optimize or adjust the leading and trailing edges of the introduced fluid. In one variation of the algorithm used to achieve a desired temperature variation of the fluid, repetitive injections of the fluid can be made and the algorithm adjusted accordingly.

Referring back to FIG. 1, an embodiment of a method of the present invention includes obtaining magnetic resonance information from the blood vessel (20). Specifically, magnetic resonance information is obtained from blood in a blood vessel. Non-limiting examples of magnetic resonance information include MR signal intensity, phase information, frequency information and any combination thereof. To obtain such magnetic resonance information, the patient is placed in a MR scanner and radiofrequency (RF) pulses are transmitted to the patient. The RF pulse sequences can be used to excite a slice, a series of slices or a volume containing the blood vessel. RF pulses can be applied in a dynamic fashion so that magnetic resonance information is measured dynamically, such as at multiple sequential points in time. For example, magnetic resonance information can be measured before, during and after the introduced fluid perfuses the blood vessel of the patient. The pulse sequences may include but are not limited to echo-planar, gradient echo, spoiled gradient echo and spin echo. For each slice, series of slices or volume, the magnetic resonance information can be spatially encoded by using magnetic field gradients including phase-encoding gradients and frequency-encoding gradients. Specifically, spatial encoding of the magnetic resonance information can be achieved by applying additional magnetic field gradients after excitation of tissue but before measurement of the magnetic resonance information (phase-encoding gradient) as well as during signal measurement (frequency-encoding gradient). In order to fully spatially encode a slice or volume of excited tissue, the excitation and measurement process can be repeated multiple times with different phase-encoding gradients. When performing a volume acquisition, two different phase encoding gradients can be applied in order to ultimately divide the volume into multiple slices. Spatial encoding allows calculation of the amount of magnetic resonance information emitted by small volume elements (voxels) in the excited slice or volume and therefore allows magnetic resonance information to be measured on a voxel-by-voxel basis in each slice, series of slices or volume.

The magnetic resonance information obtained in 20 is used to determine a magnetic resonance parameter in the blood vessel (30) according to an embodiment of a method of the present invention. Specifically, a magnetic resonance parameter of the blood of the blood vessel is determined Non-limiting examples of magnetic resonance parameters includes phase changes resulting from changes in water proton resonance frequency; changes in TI relaxation time; changes in diffusion coefficients; phase changes as determined by analysis of spectroscopic data; and any combination thereof. Methods for calculating such magnetic resonance parameters involve using well-known mathematical formulas based on the pulse sequence used and the specific parameter that is to be calculated. Methods of the present invention include measuring a single magnetic resonance parameter or multiple magnetic resonance parameters. The magnetic resonance parameter can be calculated on a voxel-by-voxel basis for each slice, series of slices or volume.

The magnetic resonance parameter determined in 30 is used to determine a temperature differential in the blood vessel (40) according to an embodiment of a method of the present invention. Specifically, a temperature differential in blood in the blood vessel is determined. Methods for calculating a temperature differential based on the above-identified magnetic resonance parameters are well-known in the art. For example, if the magnetic resonance parameter is phase changes corresponding to changes in water proton resonance frequency, a corresponding temperature differential can be calculated in accordance with the equation ΔT=Δφ(T)/αγTEB₀, where α is a temperature dependent water chemical shift in ppm (parts per million) per C⁰, γ is the gyromagnetic ratio of hydrogen, TE is the echo time; B₀ is the strength of the main magnetic field; and Δφ is phase change.

With respect to calculating a temperature differential based on changes in T1 relaxation time, changes in diffusion coefficients, or phase changes as determined by analysis of spectroscopic data such calculations can be performed, for example, in accordance with the methods described by Quesson and Kuroda (e.g. B Quesson, J A de Zwart & C T W Moonen. “Magnetic Resonance Temperature Imaging for Guidance of Thermotherapy;” 12 J Mag Res Img 525 (2000); K Kuroda, RV Mulkem, K Oshio et al. “Temperature Mapping using the Water Proton Chemical Shift; Self-referenced Method with Echo-planar Spectroscopic Imaging;” 43 Magn Reson Med 220 (2000), both of which are incorporated by reference herein. Of course, as one skilled in the art will appreciate, other methods could also be employed. Notwithstanding which magnetic resonance parameter is used to calculate a temperature differential, the measured temperature change in a voxel will correspond to the concentration of indicator (in this case heat or cold) within the voxel over time.

The temperature differential in the blood vessel is used to produce an image of the blood vessel in which a brightness or a color of pixels therein is determined by the temperature differential (50). Specifically, an image of the blood of a blood vessel is produced. Such an image can be produced by display systems following methods well-known in the art, such as the method described by C Warmuth, M Gunther & C Zimmer; “Quantification of Blood Flow in Brain Tumors: Comparison of Arterial Spin Labeling and Dynamic Susceptibility weighted Contrast-enhanced MR Imaging;” 228 Radiology 523 (2003), for example, which is incorporated by reference herein. For example, an image can be reconstructed such that the brightness of pixels in the image is determined by the magnitude of the temperature differential in the corresponding voxel. A single image or multiple images can be produced according to methods of the present invention. Images may be obtained in an axial plane, a sagittal plane, a coronal plane, an oblique plane or any combination thereof. In one example, a threshold temperature differential can be used to display flow in a vessel lumen compared with absence of flow in surrounding tissues using a fixed brightness or fixed color. In a second example, a temperature differential determined over time can be used to display flow in a vessel lumen such that a brightness or color may reflect both temperature differentials and local flow characteristics.

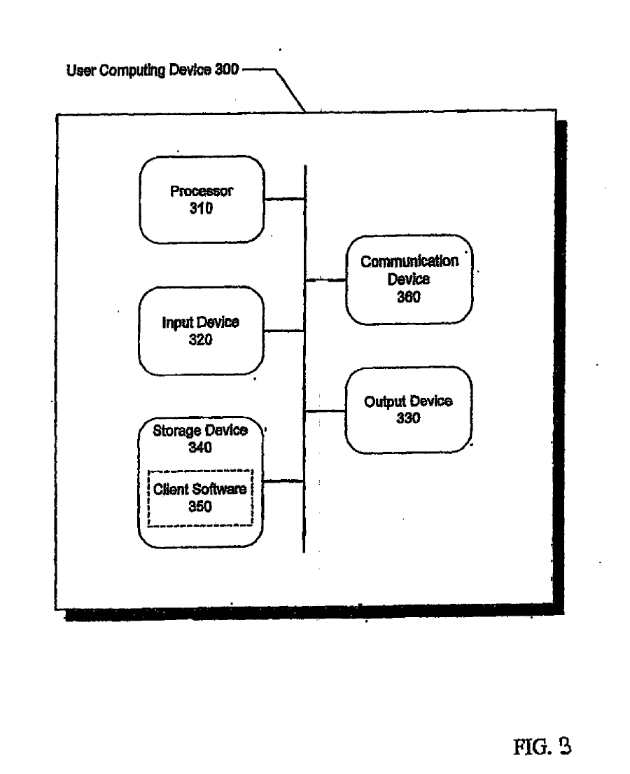

In another embodiment, the present invention provides a machine-readable medium having stored thereon a plurality of executable instructions, when executed by a processor, performs obtaining magnetic resonance information from a blood vessel of a patient after introduction of fluid into a cardiovascular system of the patient. The plurality of executable instructions further performs determining a magnetic resonance parameter in the blood vessel using the magnetic resonance information, determining a temperature differential in the blood vessel using the magnetic resonance parameter and producing an image of the blood vessel in which a brightness or a color of pixels is determined by the temperature differential. Referring to FIG. 3, the above-mentioned method may be performed by a user computing device 300 such as a MRI machine, workstation, personal computer, handheld personal digital assistant (“PDA”), or any other type of microprocessor-based device. User computing device 300 may include a processor 310, input device 320, output device 330, storage device 340, client software 350, and communication device 360. Input device 320 may include a keyboard, mouse, pen-operated touch screen, voice-recognition device, or any other device that accepts input. Output device 330 may include a monitor, printer, disk drive, speakers, or any other device that provides output. Storage device 340 may include volatile and nonvolatile data storage, including one or more electrical, magnetic or optical memories such as a RAM, cache, hard drive, CD-ROM drive, tape drive or removable storage disk. Communication device 360 may include a modem, network interface card, or any other device capable of transmitting and receiving signals over a network. The components of user computing device 300 may be connected via an electrical bus or wirelessly. Client software 350 may be stored in storage device 340 and executed by processor 310, and may include, for example, imaging and analysis software that embodies the functionality of the present invention.

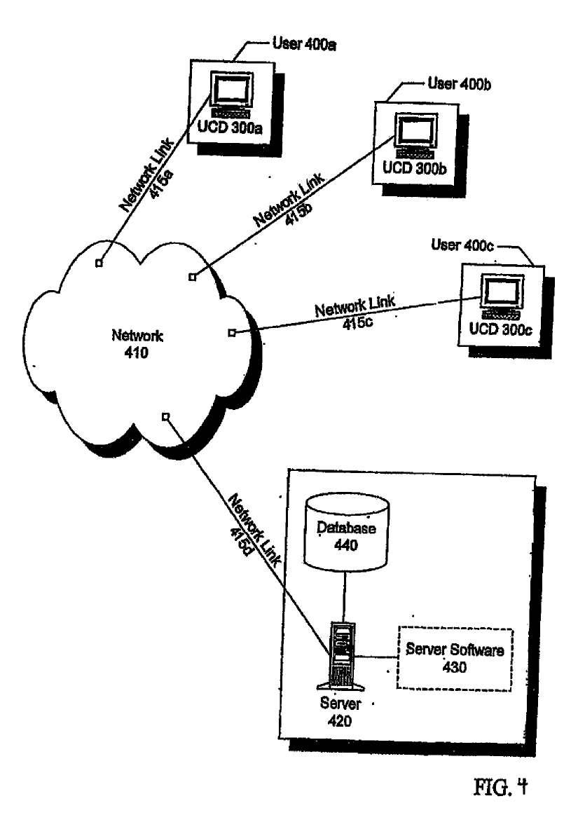

Referring to FIG. 4, the analysis functionality may be implemented on more than one user computing device 300 via a network architecture. For example, user computing device 300 may be an MRI machine that performs all determination, calculation and measurement functionalities. hi another embodiment, user computing device 300a may be a MRI machine that performs the magnetic resonance information measurement functionality and the magnetic resonance parameter determination functionality, and then transfers this determination over network 410 to server 420 or user computing device 300b or 300c for determination of a temperature differential, for example. The temperature differential could further be transferred back to user computing device 300a to produce the image of the blood vessel.

Referring again to FIG. 4, network link 415 may include telephone lines, DSL, cable networks, T1 or T3 lines, wireless network connections, or any other arrangement that implements the transmission and reception of network signals. Network 410 may include any type of interconnected communication system, and may implement any communications protocol, which may be secured by any security protocol. Server 420 includes a processor and memory for executing program instructions, as well as a network interface, and may include a collection of servers. Server 420 may include a combination of servers such as an application server and a database server. Database 440 may represent a relational or object database, and may be accessed via server 420.

User computing device 300 and server 420 may implement any operating system, such as Windows or UNIX. Client software 350 and server software 430 may be written in any programming language, such as ABAP, C, C++, Java or Visual Basic

The foregoing description has been set forth merely to illustrate the invention and are not intended as being limiting. Each of the disclosed aspects and embodiments of the present invention may be considered individually or in combination with other aspects, embodiments, and variations of the invention. In addition, unless otherwise specified, none of the steps of the methods of the present invention are confined to any particular order of performance. Modifications of the disclosed embodiments incorporating the spirit and substance of the invention may occur to persons skilled in the art and such modifications are within the scope of the present invention. Furthermore, all references cited herein are incorporated by reference in their entirety.

What is claimed is:

1. A method for producing an image of a blood vessel of a patient comprising:
introducing a fluid into a cardiovascular system of the patient;
obtaining magnetic resonance information from the blood vessel;
determining a magnetic resonance parameter in the blood vessel using the magnetic resonance information;
determining a temperature differential in the blood vessel using the magnetic resonance parameter; and
producing an image of the blood vessel in which a brightness or a color of pixels therein is determined by the temperature differential.

2. The method of claim 1, wherein the blood vessel is an artery.

3. The method of claim 1, wherein the blood vessel is a vein.

4. The method of claim 1, wherein the fluid is a saline solution.

5. The method of claim 1, wherein the cardiovascular system is a peripheral or a central vein.

6. The method of claim 1, wherein the cardiovascular system is a central or a peripheral artery.

7. The method of claim 1, wherein introducing the fluid comprises introducing the fluid at a temperature that is below body temperature of the patient.

8. The method of claim 1, wherein obtaining the magnetic resonance information comprises:
placing the patient in a magnetic resonance scanner;
transmitting radiofrequency pulses to the patient to excite a slice, a series of slices or a volume containing the blood vessel; and
measuring the magnetic resonance information from the blood vessel.

9. The method of claim 1, wherein obtaining magnetic resonance information comprises collecting the magnetic resonance information at multiple sequential points in time from the blood vessel after introducing the fluid.

10. The method of claim 9, wherein collecting the magnetic resonance information at multiple sequential points comprises collecting the magnetic resonance information before, during and after the introduced fluid perfuses the blood vessel of the patient.

11. The method of claim 1, wherein determining the magnetic resonance parameter comprises determining the magnetic resonance parameter on a voxel-by-voxel basis through the blood vessel of the patient.

12. The method of claim 1, wherein the magnetic resonance parameter comprises changes in water proton resonance frequency and the temperature differential is determined using the changes in water proton resonance frequency.

13. The method of claim 1, wherein the magnetic resonance parameter comprises changes in T1 relaxation time of water protons and the temperature differential is determined using the changes in T1 relaxation time.

14. The method of claim 1, wherein the magnetic resonance parameter comprises changes in a diffusion coefficient of water in the blood vessel and the temperature differential is determined using the changes in a diffusion coefficient.

15. The method of claim 1, wherein the magnetic resonance parameter comprises changes in magnetic resonance spectroscopy measurements of the blood vessel and the temperature differential is determined using the changes in magnetic resonance spectroscopy measurements.

16. A method for producing an image of a blood vessel of a patient comprising:
introducing a gas into a lung of the patient;
obtaining magnetic resonance information from the blood vessel;
determining a magnetic resonance parameter in the blood vessel using the magnetic resonance information;
determining a temperature differential in the blood vessel using the magnetic resonance parameter; and
producing an image of the blood vessel in which a brightness or a color of pixels therein is determined by the temperature differential.

17. A machine-readable medium having stored thereon a plurality of executable instructions, which, when executed by a processor, perform the following:
obtaining magnetic resonance information from a blood vessel of a patient after introduction of a fluid into a cardiovascular system of the patient;
determining a magnetic resonance parameter in the blood vessel using the magnetic resonance information;
determining a temperature differential in the blood vessel using the magnetic resonance parameter; and
producing an image of the blood vessel in which a brightness or a color of pixels therein is determined by the temperature differential.

18. The machine-readable medium of claim 17, wherein determining a magnetic resonance parameter in the blood vessel comprises measuring the magnetic resonance information on a voxel-by-voxel basis.

19. The machine-readable medium of claim 17, wherein obtaining the magnetic resonance information comprises obtaining the magnetic resonance information before, during and after blood perfuses the blood vessel.

20. The machine-readable medium of claim 17, wherein the magnetic resonance parameter comprises changes in water proton resonance frequency and the temperature differential is determined using the changes in water proton resonance frequency.

21. A system for producing an image of a blood vessel of a patient comprising:
means for introducing a fluid into a cardiovascular system of the patient;
means for obtaining magnetic resonance information from the blood vessel;
means for determining a magnetic resonance parameter in the blood vessel using the magnetic resonance information;
means for determining a temperature differential in the blood vessel using the magnetic resonance parameter; and
means for producing an image of the blood vessel in which a brightness or a color of pixels therein is determined by the temperature differential.

22. The system of claim 21, wherein the means for introducing a fluid comprise a central arterial catheter.

23. The system of claim 21, wherein the means for introducing a fluid comprises a central venous catheter.

24. The system of claim 21, wherein the means for introducing a fluid comprises a peripheral venous catheter.

25. The system of claim 21, wherein the means for determining a temperature differential comprises means for calculating changes in water proton resonance frequency and using the changes in water proton resonance frequency to determine the temperature differential.
